(19) United States
(12) Patent Application Publication
(10) Pub. No.: US 2026/0363614 A1
(43) Pub. Date: Nov. 19, 2026

(54) SYSTEMS, METHODS, AND MEDIA FOR MOBILE DETECTION OF A MEDICAL CONDITION AND WIRELESS ACTUATION OF A DRUG DELIVERY DEVICE

(71) Applicant: Purdue Research Foundation, West Lafayette, IN (US)

(72) Inventors: Hyowon LEE, West Lafayette, IN (US); Michael MCLEAN, West Lafayette, IN (US)

(21) Appl. No.: 19/475,691

(22) PCT Filed: May 6, 2024
(86) PCT No.: PCT/US2024/027995

§ 371 Date: Oct. 15, 2025

Related U.S. Application Data
(60) Provisional application No. 63/500,465, filed on May 5, 2023.

(51) Int. Cl. A61B 5/00; A61B 5/145; A61B 5/1455

(52) U.S. Cl. CPC A61B 5/4839; A61B 5/14546; A61B 5/14552; A61B 5/6824; A61B 5/7264; A61B 5/7282; A61B 2560/0214; A61B 2560/0462; A61B 2562/0233

(57) ABSTRACT

Methods, systems, and media for detecting mobile detection of a medical condition and wireless actuation of a drug delivery device are provided. In some embodiments, the system comprises: a power supply; a physiological sensor; a trigger switch; a signal generator; a transmitting coil electrically coupled to the power supply via the trigger switch and the signal generator; and at least one processor that is programmed to: receive signals from the physiological sensor; determine that a triggering event has occurred based on the signals received from the physiological sensor; in response to determining that the triggering event has occurred, cause the trigger switch to provide power to the signal generator, such that power is transmitted by the transmitting coil to wirelessly activate the drug delivery device.

### CROSS-REFERENCE TO RELATED APPLICATIONS

This application claims priority to U.S. Provisional Application No. 63/500,465, filed on May 5, 2023, the entirety of which is incorporated herein by reference.

### STATEMENT REGARDING FEDERALLY SPONSORED RESEARCH

N/A

### BACKGROUND

Opioid-related deaths in the United States (US) rose to 48,602 in 2018, followed by another sharp increase to 68,620 in 2020, which represents more than one third of the entire drug overdose deaths that year. Prescriptive analgesics (e.g., codeine, fentanyl), and heroin are the most common opioids related to overdose mortality. Physicians use opioids for pain management, especially for those patients suffering from severe illnesses. However, the misuse of opioids roughly affects vital physiological processes in the central nervous system, leading to devastating hypoxic events and severe cardiorespiratory depression.

A current treatment to address an opioid overdose is to administer a burst dose of naloxone, a Federal Drug Administration (FDA) approved strong opiate antagonist that counteracts the physiological effects of an opioid-induced cardiorespiratory depression event. Naloxone is an indispensable harm reduction tool in efforts to reduce opioid-related deaths. Most commonly, naloxone has been shown to be effective when administered via nasal inhalation, intramuscular injection, or subcutaneous injection.

To reverse the life-threatening effects of an opioid overdose, it is crucial to deliver the naloxone as quickly as possible. However, a person experiencing an opioid overdose is often found unconscious and under cardiorespiratory attack, limiting the person's ability to properly self-administer the life-saving medicine, even if the person is carrying or can otherwise easily access naloxone.

Accordingly, systems, methods, and media for detecting mobile detection of a medical condition and wireless actuation of a passive or an active drug delivery device are desirable.

### SUMMARY

In accordance with some embodiments of the disclosed subject matter, systems, methods, and media for mobile detection of a medical condition and wireless actuation of a drug delivery device are provided.

In accordance with some embodiments of the disclosed subject matter, a system for mobile detection of a medical emergency and wireless activation of a drug delivery device is provided, the system comprising: a power supply; a physiological sensor; a trigger switch; a signal generator; a transmitting coil electrically coupled to the power supply via the trigger switch and the signal generator; and at least one processor that is programmed to: receive signals from the physiological sensor; determine that a triggering event has occurred based on the signals received from the physiological sensor; in response to determining that the triggering event has occurred, cause the trigger switch to provide power to the signal generator, such that power is transmitted by the transmitting coil to wirelessly activate the drug delivery device.

In some embodiments, the power supply comprises at least one battery, and is configured to provide power at about 7.4 volts.

In some embodiments, the physiological sensor comprises a near-infrared spectroscopy (NIRS) sensor.

In some embodiments, the signal generator comprises a zero-voltage switching (ZVS) circuit.

In some embodiments, the transmitting coil comprises a pancake coil.

In some embodiments, the transmitting coil comprises a polyamide-insulated copper magnetic wire.

In some embodiments, the signals comprise values output by a photodetector of the physiological sensor.

In some embodiments, the at least one processor is further programmed to: estimate oxyhemoglobin (HbO₂) and deoxyhemoglobin (HbR) based on the values.

In some embodiments, the at least one processor is further programmed to: estimate a variable R that is indicative of oxygen saturation (SpO₂) based on the values.

In some embodiments, the signals comprise oxyhemoglobin (HbO₂) and deoxyhemoglobin (HbR) values output by the physiological sensor.

In some embodiments, the signals comprise a variable R output by the physiological sensor.

In accordance with some embodiments of the disclosed subject matter, a method for mobile detection of a medical emergency and wireless activation of a drug delivery device is provided, the method comprising: receiving signals from a physiological sensor; determining that a triggering event has occurred based on the signals received from the physiological sensor; in response to determining that the triggering event has occurred, causing a trigger switch to provide power to a signal generator, such that power is transmitted by a transmitting coil to wirelessly activate the drug delivery device.

In accordance with some embodiments of the disclosed subject matter, a non-transitory computer readable medium containing computer executable instructions that, when executed by a processor, cause the processor to perform a method for mobile detection of a medical emergency and wireless activation of a drug delivery device is provided, the method comprising: receiving signals from a physiological sensor; determining that a triggering event has occurred based on the signals received from the physiological sensor; in response to determining that the triggering event has occurred, causing a trigger switch to provide power to a signal generator, such that power is transmitted by a transmitting coil to wirelessly activate the drug delivery device.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows an example of a system for mobile detection of a medical condition and wireless actuation of a drug delivery device in accordance with some embodiments of the disclosed subject matter.

FIGS. 2A and 2B show an example of hardware that can be used to implement a wearable device in accordance with some embodiments of the disclosed subject matter.

FIG. 3 shows an example of hardware that can be used to implement a light emitting diode (LED) driving circuit in accordance with some embodiments of the disclosed subject matter.

FIG. 4 shows an example of hardware that can be used to implement a transimpedance amplifier in accordance with some embodiments of the disclosed subject matter.

FIG. 5 shows an example of a process for mobile detection of a medical condition and wireless actuation of a drug delivery device in accordance with some embodiments of the disclosed subject matter.

FIG. 6 shows an example of components implemented for a benchtop experiment to test functionality of a wearable device implemented in accordance with some embodiments of the disclosed subject matter.

FIG. 7 shows an example of a setup for a human experiment to test capabilities of a prototype wearable device implemented in accordance with some embodiments of the disclosed subject matter.

FIGS. 8A-F shows an example of components of the prototype wearable device shown in FIG. 7, and the prototype wearable device worn by two different subjects.

FIGS. 9A-E shows an example of a setup for a benchtop experiment to test to test functionality of the prototype wearable device shown in FIG. 7.

FIGS. 9F-G show results of tests with the benchtop experiment setup of FIGS. 9A-E.

FIG. 10 shows examples of peak-peak values of infrared and red intensity recorded during the human experiment described in connection with FIG. 7.

FIG. 11 shows examples of averaged R modulation index values recorded during the human experiment described in connection with FIG. 7.

FIG. 12 shows examples of peak-peak values of oxyhemoglobin and deoxyhemoglobin changes calculated during the human experiment described in connection with FIG. 7.

FIG. 13 shows a table that includes a summary of p-values for the analysis on brachial occlusion during the human experiment described in connection with FIG. 7.

FIG. 14 shows a table that includes a summary of p-values for the analysis on breath hold during the human experiment described in connection with FIG. 7.

### DETAILED DESCRIPTION

In accordance with various embodiments, mechanisms (which can, for example, include systems, methods, and media) for mobile detection of a medical condition and wireless actuation of a drug delivery device are provided.

In some embodiments, mechanisms described herein can be used to implement a sensor capable of rapidly detecting an opioid-induced respiratory depression. Additionally, in some embodiments, mechanisms described herein can be used to implement a closed-loop system that can deliver a burst amount of naloxone in response to detection of an opiod-induced respiratory depression, serving as a tool to keep an opioid user alive until specialized medical care can be provided, especially for patients residing in rural areas and places with low availability of healthcare centers.

As described below, mechanisms described herein were used to design, fabricate, and evaluate a prototype wrist-wearable device for sensing change in oxygenation level and actuating a drug deliver capsule based on three well-defined components. In some embodiments, a wearable device for mobile detection of a medical condition (e.g., an opioid-induced low oxygen level) and wireless actuation of a drug delivery device can include various components, such as: (i) a near-infrared spectroscopy (NIRS) sensor, (ii) a metal-oxide semiconductor field-effect transistor (MOSFET) dual switch, and (iii) a modified zero-voltage switching (ZVS) circuit. Experiments described below demonstrate the capability of an NIRS system to detect changes in oxyhemoglobin (HbO₂) and deoxyhemoglobin (HbR) during induced low oxygenation events, considering the integration of an approach based on a modulation index to calculate SpO₂ levels. Additionally, experiments described below revealed the ability of a system implemented in accordance with mechanisms described herein to rapidly release a dose of life-saving medication (e.g., naloxone) from a minimally invasive capsule for a rapid response during an opioid overdose. In some embodiments, analog-digital filters and signal data processing resources can be incorporated into a device implemented in accordance with mechanisms described herein to reduce noise induced by user movements and the most relevant sources of artifacts that worsen the signal quality of the NIRS system. Additionally or alternatively, in some embodiments, the device can be further miniaturized and the electronics can be further optimized, reducing the size of the device as much as possible to improve usability. In some embodiments, mechanisms described herein can be used to help mitigate the devastatingly deadly rates of opioid overdose and in other potential emergency life-threatening drug delivery applications. In some embodiments, mechanisms described herein can be used to facilitate rapid deployment of subcutaneous medication to treat other medical events, such as anaphylaxis (e.g., with low oxygenation being an indicator of severe anaphylaxis), pediatric nocturnal hypoglycemia (e.g., using a sensor(s) configured detect blood sugar levels, in addition to, or in lieu of, oxygenation).

The development of wearable devices for the detection of opioid overdose and naloxone drug delivery is a promising harm-reduction technology to address mortality associated with the opioid epidemic. A wrist-wearable system referred to as the Q-sensor was the first wrist-wearable system capable of measuring physiological changes including skin electrodermal activity (EDA), skin temperature, and 3D locomotion to establish correlations with an opioid-overdose event. However, further studies showed that EDA and locomotion were not strongly statistically valid to confirm correlations with an opioid-related drug overdose. Recently, a closed-loop proof-of-concept wearable injector system of naloxone was developed. The wearable system measured opioid-induced apnea events and respiratory depression, implementing a pair of body accelerometers and administering naloxone through a commercial injector system. However, the system was relatively bulky and can be easily perceived through a wearer's garments. Additionally, the invasiveness of having an injector system may increase the risk of infections and other side effects.

In some embodiments, mechanisms described herein can utilize optical sensors to detect opioid-induced apnea and cardiorespiratory depression events. For example, photoplethysmography (PPG) can be used as a noninvasive approach that uses the absorbance property of oxygenated and deoxygenated hemoglobin to calculate the respiratory rate and collect electrocardiographic information. PPG can be implemented using a light transmitter to propagate through the tissue and a photodetector to perceive changes in the emitted light. Using PPG, opioid-induced cardiorespiratory events were detected with 90% accuracy in one study. A wrist-mounted PPG biosensor referred to as Empatica E4 was implemented for the detection and recovery process of opioid-related overdose use. In some embodiments, mechanisms described herein can use a subset of PPG, such as NIRS, which is an optical technique that can be used to detect cardiopulmonary activity of the body using red and infrared LEDs. Using a relationship between the absorbance of oxyhemoglobin (HbO₂) and deoxyhemoglobin (HbR), it is possible to estimate a crucial parameter notated as the modulation index (R) to determine the peripheral capillary oxygen saturation levels (SpO₂). Although PPG and NIRS share similar instrumentation, NIRS relies on simple mathematical calculations and assumptions to obtain cardiorespiratory information.

In some embodiments, mechanisms described herein were used to design, fabricate, and evaluate a prototype wearable device with a closed-loop to detect a medical event (e.g., opioid-induced cardiorespiratory depression) and to deliver a burst of medication (e.g., naloxone). Such a system can be used to implement a fully wearable device based on NIRS. As described below, in some embodiments, a wrist-wearable device for sensing change in oxygenation level can include three stages: (i) a sensor based on NIRS technology to detect cardiopulmonary depression events; (ii) a MOSFET switch to activate the actuator; and (iii) an electromagnetic field generator based on the Zero Voltage Switching (ZVS) circuit to deliver the live-saving antidote as quickly as possible.

Opioid overdose is a prominent public health issue with devastating consequences that can be prevented and mitigated through the invention of novel technologies and engineered solutions. Herein, a closed-loop wearable device for the detection of cardiorespiratory depression and the delivery of the life-saving medication (naloxone) is described. A wrist wearable device has the potential to improve usability with more compact electronics, specifically in terms of size reduction of at least 26% approximately for the electromagnetic field generator than a previous technology. As described below, mechanisms described herein can include three well-defined electronic stages: (i) a NIRS sensor for the detection of an induced low peripheral oxygenation event, (ii) a MOSFET dual switch to couple the instrumentation circuit to the electromagnetic field generator, and (iii) a modified compact ZVS electromagnetic field generator circuit, serving as an actuator to activate the delivery process of naloxone through a minimally invasive implantable capsule (e.g., as described in Dhowan et al., “Simple minimally-invasive automatic antidote delivery device (a2d2) towards closed-loop reversal of opioid overdose,” Journal of Controlled Release, 306:130-137 (2019), and in Lee et al., U.S. Pat. No. 11,439,747, which is hereby incorporated by reference herein in its entirety

In a previously proposed closed-loop system with a wearable injector system, based on the detection of apneic motion and cardiorespiratory events, some limitations were denotated, specifically in the size of the device and how it might affect the willingness of opioid users to wear such a conspicuous wearable device due to stigma associated with opioid use disorder (OUD). The popularization of wrist-worn consumer electronics devices can be expected to alleviate potential concerns of wearability and increase the compliance of OUD patients.

In some embodiments, mechanisms described herein can be used implement a reliable closed-loop fully wearable device to detect an opioid overdose-induced cardiorespiratory event, capable of delivering a burst dose of naloxone as quickly as possible. Results described below showed that the electromagnetic actuator is capable of releasing the drug within 12.28±1.38 seconds(s) at 1 centimeter (cm) away when a corresponding remotely actuated drug reservoir is implanted under the skin.

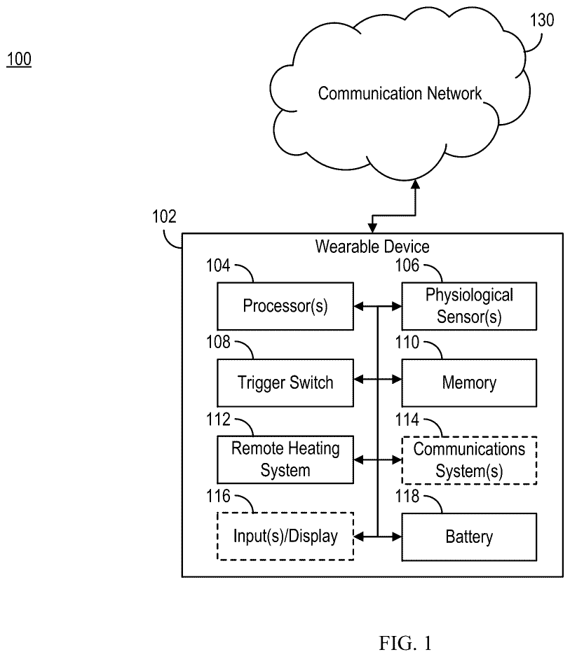

FIG. 1 shows an example of a system for mobile detection of a medical condition and wireless actuation of a drug delivery device in accordance with some embodiments of the disclosed subject matter.

As shown in FIG. 1, a wearable device 102 can be configured to perform any suitable functions described herein, such as monitoring one or more physiological signals, detecting onset of a medical emergency (e.g., an emergency that indicates a timely dose of a drug or other therapeutic is needed), activation of a remote heating system, etc.

In some embodiments, wearable device 102 can communicate with one or more other devices over a communication network 130. In some embodiments, such communications can be received from any suitable device, such as a remote physiological sensor, a server, a smartphone, a tablet computer, etc., and/or sent to any suitable device, such as a remote physiological sensor, a server, a smartphone, a tablet computer, etc.

As shown in FIG. 1, in some embodiments, wearable device 102 can include a processor 104, one or more physiological sensors 106, a trigger switch 108, memory 110, a remote heating system 112, one or more communication systems 114, one or more inputs and/or displays 116, and/or one or more power sources 118 (e.g., one or more batteries).

In some embodiments, processor 104 can be any suitable hardware processor or combination of processors, such as a microcontroller, a central processing unit (CPU), an accelerated processing unit (APU), a graphics processing unit (GPU), a field programmable gate array (FPGA), an application specific integrated circuit (ASIC), etc.

In some embodiments, physiological sensor(s) 106 can include any suitable physiological sensor or combination of physiological sensors, such as a NIRS sensor (e.g., utilizing IR and red light), a PPG sensor (e.g., using green and/or red light). In some embodiments, processor 104 can be configured to control physiological sensor(s) 106, for example, by controlling operation of physiological sensor(s) 106 and/or controlling an operational state of physiological sensor(s) 106. For example, processor 104 can be configured to provide control signals to physiological sensor(s) 106. As another example, processor 104 can be configured to control an operational state of physiological sensor(s) 106, and physiological sensor(s) 106 can include logic and/or circuits that control operation of physiological sensor(s) 106.

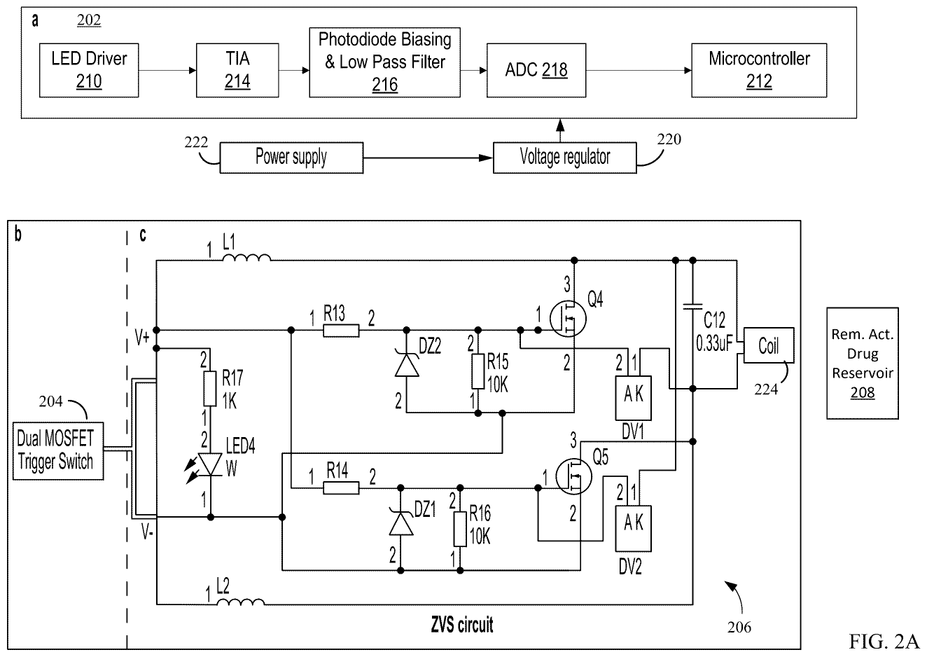

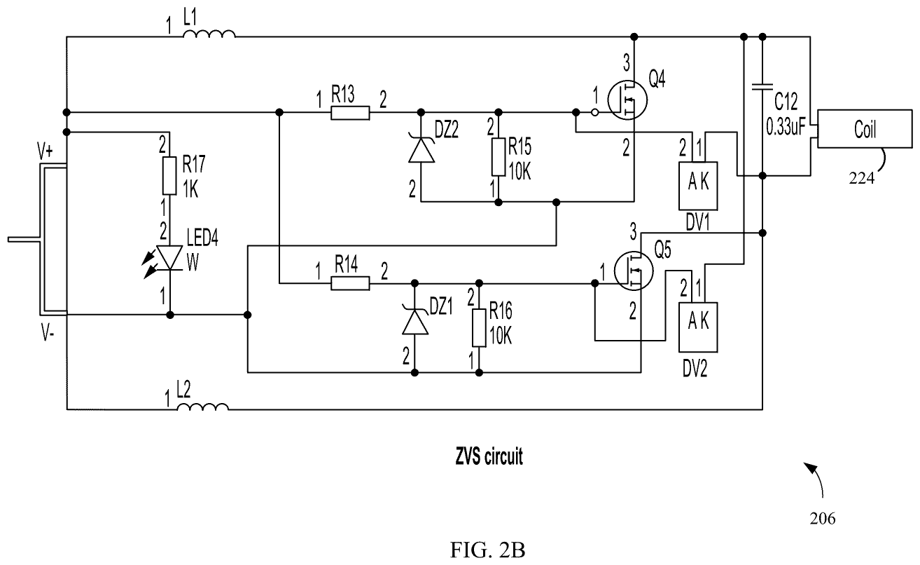

In some embodiments, trigger switch 108 can include any suitable logic, hardware, and/or software configured to activate a remote heating system 112 in response to a signal from processor 104 and/or physiological sensor(s) 106. For example, as described below in connection with FIG. 2A, trigger switch 108 can be configured to provide power to a ZVS circuit in response to a signal from processor 104 (e.g., implemented with a microcontroller).

In some embodiments, memory 110 can include any suitable storage device or devices that can be used to store instructions, values, etc., that can be used, for example, by processor 104 to control operation of wearable device 102, record physiological signals and/or physiological data (e.g., HbO₂, HbR, SpO₂, etc.), to communicate with another device via communications system(s) 114, etc. Memory 110 can include any suitable volatile memory, non-volatile memory, storage, or any suitable combination thereof. For example, memory 110 can include RAM, ROM, EEPROM, one or more flash drives, one or more hard disks, one or more solid state drives, one or more optical drives, etc. In some embodiments, memory 110 can have encoded thereon a program (e.g., a computer program) for controlling operation of wearable device 102. In such embodiments, processor 104 can execute at least a portion of the program to record physiological signals, physiological data, receive information from another device, transmit information to another device, etc.

In some embodiments, remote heating system 112 can include any suitable components capable of transmitting power to a remotely actuated drug reservoir that is in relatively close proximity to remote heating system 112 (e.g., within about 1 cm when the remotely actuated drug reservoir is implanted under the skin of a human). In some embodiments, remote heating system 112 can include magnetic field generator and/or high frequency amplifier, such as a ZVS circuit coupled between trigger switch 108 and a coil configured to wirelessly transmit power to the remotely actuated drug reservoir. In some embodiments, remote heating system 112 can be electrically coupled to power source 118 via trigger switch 108, such that remote heating system 112 is electrically isolated from power source 118 while trigger switch 108 is in an open state.

In some embodiments, communications systems 114 can include any suitable hardware, firmware, and/or software for communicating information over communication network 130 and/or any other suitable communication networks. For example, communications systems 114 can include one or more transceivers, one or more communication chips and/or chip sets, etc. In a more particular example, communications systems 114 can include hardware, firmware and/or software that can be used to establish a Bluetooth connection, Wi-Fi connection, a cellular connection, a universal service bus (USB) connection, an Ethernet connection, etc.

In some embodiments, input(s)/display(s) 116 can include any suitable input device(s) and/or sensor(s) that can be used to receive user input, such as one or more buttons, a touchscreen, a microphone, etc. Additionally or alternatively, in some embodiments, input(s)/display(s) 116 can include any suitable display device(s), such as a touchscreen, one or more indicator lights (e.g., implemented with LEDs) to signal an operational and/or power status of wearable device 102, etc.

In some embodiments, power supply 118 can include any suitable source of power and/or any suitable power electronics to provide adequate power for operation of other components of wearable device 102. For example, power supply 118 can include one or more batteries, a voltage regulator, etc.

In some embodiments, communication network 130 can be any suitable communication network or combination of communication networks. For example, communication network 130 can include a peer-to-peer network (e.g., a Bluetooth network), a Wi-Fi network (which can include one or more wireless routers, one or more switches, etc.), a cellular network (e.g., a 3G network, a 4G network, a 5G network, etc., complying with any suitable standard, such as CDMA, GSM, LTE, LTE Advanced, 5G NR, etc.), a wired network, etc. In some embodiments, communication network 130 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as Bluetooth links, Wi-Fi links, cellular links, wired links, fiber optic links, etc.

FIGS. 2A and 2B show an example of hardware that can be used to implement a wearable device (e.g., the wearable device 102) in accordance with some embodiments of the disclosed subject matter.

FIG. 2A shows an electronic drug delivery system 200 in accordance with some embodiments of the disclosed subject matter. As depicted in FIG. 2A, three electronic systems of the wearable device or drug delivery system 200 may include: an NIRS sensor 202, a MOSFET switch 204, and an electromagnetic ZVS actuator 206. That is, FIG. 2A, panel (a) shows a block diagram of principal electronic elements of the NIRS sensor 202; FIG. 2A, panel (b) shows a block description of the MOSFET switch 204 (e.g., a Dual MOSFET trigger switch 204); and FIG. 2A, panel (c) shows a circuit design of a ZVS actuator 206 (e.g., a ZVS electromagnetic field generator 206, also referred to as a ZVS driver or ZVS circuit). In the illustrated example, system 200 also includes a remotely actuated drug reservoir 208. The remotely actuated drug reservoir 208 may be a passive drug delivery device, an active drug delivery device, or a combination of a passive and active drug delivery device (e.g., controllable as a passive device and also as an active device). With reference to both FIGS. 1 and 2A, the NIRS sensor 202 may be an example of the physiological sensor(s) 106, the microcontroller 212 may be an example of the processor 104 (e.g., the microcontroller 212 may serve as both the processor 104 and also as part of the NIRS sensor 202), the power supply 222 may be an example of the battery 118, the dual MOSFET trigger switch 204 may be an example of the trigger switch 108, and the ZVS actuator 206 (including coil 224) may be an example of the remote heating system 112.

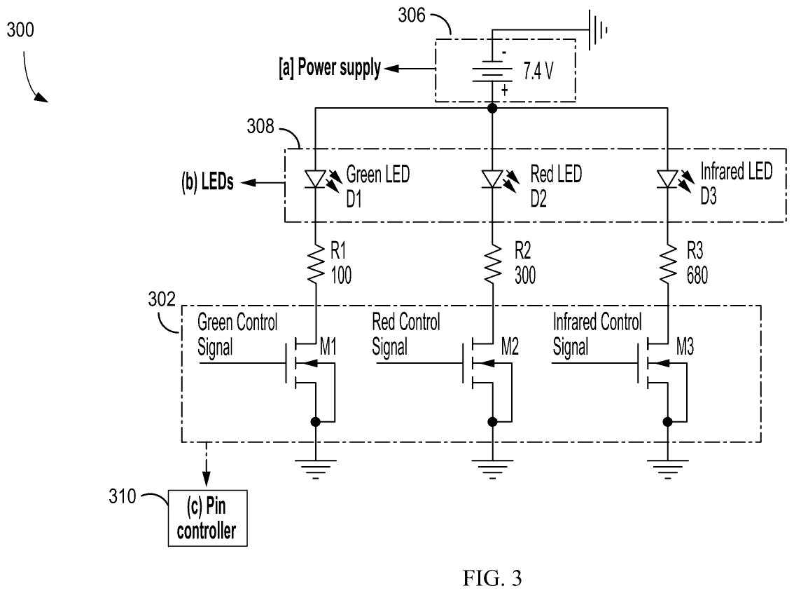

FIG. 2A, panel (a) illustrates electronic components involved in an implementation of the NIRS sensor 202. The SFH7050 (available from OSRAM Opto Semiconductors), which includes three LEDs with specific wavelengths (λ=525 nm, λ=660 nm, λ=950 nm) can be used to serve as the light source of the device, and a photodiode can be used to detect light. In some embodiments, each LED can be pulsed out of phase (e.g., using LED driver 210). In a particular example, each LED can be pulsed out of phase at a frequency of 14.05 hertz (Hz)), establishing an on-time pulse of 15 milliseconds (ms) to yield a duty cycle of 0.21. The LEDs may be part the LED driver 210, as illustrated with respect to FIG. 3. The logic of the system or NIRS sensor 202 can be handled using a microcontroller 212 (e.g., a SAMD21G18AU microcontroller available from Microchip Technology, Inc.). Two analog components of the NIRS sensor can include: a light-emitting diode (LED) driver 210 and a transimpedance amplifier (TIA) 214. Further supporting systems may include, for example, a low-pass filter 216, an analog-to-digital converter (ADC) 218, a voltage regulator 220, a power supply 222, and miscellaneous sub-circuits to facilitate a suitable operation of the system.

To properly pair the sensing and instrumentation circuit (low-current system) and meet the actuator power requirements (electromagnetic field generator), a commercial dual high-power SWITCH 204 (e.g., implemented using a dual MOSFET Tigger Switch 0-20 kilohertz (kHz) PMW, procured from Amazon.com, Inc) can be implemented. The switch can be connected to the ZVS circuit 206 and activated by the microcontroller 212 as illustrated in FIG. 2A.

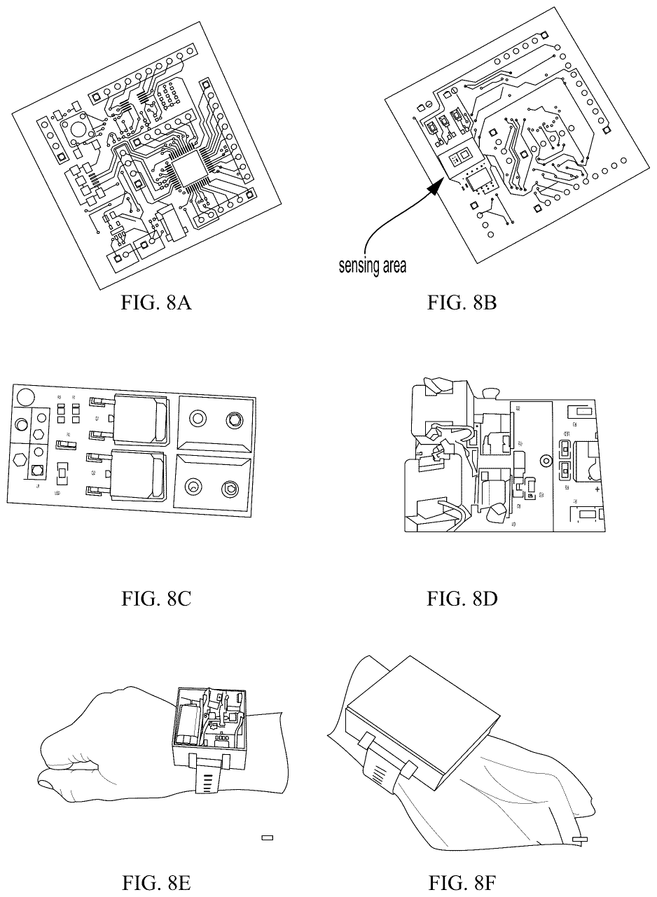

In some embodiments, a compact version of a commercial ZVS (e.g., a Yooso ZVS Driver Circuit, procured from Amazon.com, Inc) can be implemented. The circuit can be based on the driver electromagnetic field generator implemented in Dhowan et al., “Simple minimally-invasive automatic antidote delivery device (a2d2) towards closed-loop reversal of opioid overdose,” Journal of Controlled Release, 306:130-137 (2019), in order to reduce the size of the device as much as possible. In some embodiments, a ZVS circuit 206 implemented herein can incorporate smaller choke inductors, which can be used to block high-frequency voltage signals. At the same time, this family of inductors allows direct current (DC) and low alternating current (AC) frequencies to pass while maintaining the properties of the original signal. FIG. 2A, panel (c) and FIG. 2B show the implemented electronic circuit of the ZVS driver 206. The printed circuit board (PCB) shown in FIG. 8D was designed with licensed specialized program (from Altium) and manufactured by JLCPCB, Inc. In some embodiments, a coil 224 used to transmit energy to heat the implanted drug reservoir 208 can include a 9 turn pancake copper coil, implemented using a commercial polyamide-insulated magnetic wire (for example, a Magnet Wire MW35-C HY), which can improve ZVS performance in terms of heat transfer. The inductance of the package coil was measured using a commercial LCR meter (an IM3535 LCR meter available from Hioki).

In some embodiments, to ensure proper isolation between the three above-described electronic components of the system and avoid undesirable short circuits within the case, a 1 millimeter (mm) layer of Ecoflex 00-30 can be placed over the top surface of the circuits. In some embodiments, all the elements can be packed inside a wrist case (e.g., as described below to be used for the benchtop experiments).

In some embodiments, sample data can be acquired with the 14.05 Hz sampling rate of the LEDs using the microcontroller 212 and the ADC 218 (e.g., an ADS115 16-bit resolution analog-to-digital converter, available from Texas Instruments, Inc). In some embodiments, only 15 bits of the ADC 218 can be used due to a slight drop in the negative values of the signal, resulting in a minor modification of 0.1 mV resolution on a 3.3 V instrumentation-powered system. For the benchtop experiment described below, the acquired data was saved as a text file for further processing in Python. In some embodiments, the acquired data can be stored in memory and/or provided to a processor (e.g., a microcontroller) for processing to determine physiological signals (e.g., HbO₂ and HbR) and/or to determine whether a wearer is experiencing a medical emergency.

Due to the limitation of current analog circuits to filter data from NIRS devices concerning low peripheral oxygenation applications, for the benchtop experiments the raw data was acquired from the wearable device and digitally processed on a computer. However, in practice, the raw data can be processed on a processor within the device (e.g., by the microcontroller 212).

In some embodiments, processing of the NIRS data can include any suitable processing techniques. For example, in some embodiments, three principal filters can be applied to process the data. First, a fifth order finite impulse response (FIR) low pass filter can be applied to smooth the raw data. The filter can be configured for a fₛₜₒₚ=2.0 Hz and fₚₐₛₛ=1.5 Hz (least squares) with equally weighted passband and stopband. Second, a third order infinite impulse response (IIR) Butterworth filter can be applied with a cutoff frequency (fc) of 0.25 Hz to eliminate the low frequency movement effects and the DC offset. The peak-to-peak voltages values for a 40-sample epoch can be extracted and placed into an array. Finally, to smooth the final signal in terms of further fluctuations caused by rapid and high-frequency movements, a five-sample sliding medial filter can be applied.

In some embodiments, to estimate oxygen saturation (SpO₂) levels, both AC and DC data from the red and infrared color channels can be used, calculating the intermediate variable R using EQ. (1).

$$R = \frac{{I}_{A C , red} / {I}_{D C , red}}{{I}_{A C , IR} / {I}_{D C , IR}} \qquad ( 1 )$$

In some embodiments, R can be used as a proxy for SpO₂, for example using a suitable reference curve.

In some embodiments, to estimate the HbR and HbO₂ levels, a modified version of the Beer-Lambert Law with scattering approach can be used. EQS. (2) and (3) represent this modified mathematical model.

$$Δ [ HbR ] = \frac{{ϵ}_{{HbO}_{2}} ( {λ}_{2} ) \frac{- ln ( \frac{{I}_{t} ( {λ}_{1} )}{{I}_{t - 1} ( {λ}_{1} )} )}{DPF ( {λ}_{1} )} - {ϵ}_{{HbO}_{2}} ( {λ}_{1} ) \frac{- ln ( \frac{{I}_{t} ( {λ}_{2} )}{{I}_{t - 1} ( {λ}_{2} )} )}{DPF ( {λ}_{2} )}}{{ϵ}_{HbR} ( {λ}_{1} ) {ϵ}_{{HbO}_{2}} ( {λ}_{2} ) - {ϵ}_{{HbO}_{2}} ( {λ}_{1} ) {ϵ}_{HbR} ( {λ}_{2} )} \qquad ( 2 )$$

$$Δ [ {HbO}_{2} ] = \frac{{ϵ}_{HbR} ( {λ}_{2} ) \frac{- ln ( \frac{{I}_{t} ( {λ}_{2} )}{{I}_{t - 1} ( {λ}_{2} )} )}{DPF ( {λ}_{2} )} - {ϵ}_{HbR} ( {λ}_{2} ) \frac{- ln ( \frac{{I}_{t} ( {λ}_{1} )}{{I}_{t - 1} ( {λ}_{1} )} )}{DPF ( {λ}_{1} )}}{{ϵ}_{HbR} ( {λ}_{1} ) {ϵ}_{{HbO}_{2}} ( {λ}_{2} ) - {ϵ}_{{HbO}_{2}} ( {λ}_{1} ) {ϵ}_{HbR} ( {λ}_{2} )} \qquad ( 3 )$$

In some embodiments, HbO₂ or HbR can be used alone, or in combination. In some embodiments, using both measurements can provide redundancy and/or improve accuracy of detecting a low oxygenation state.

FIGS. 2A and 2B illustrate and are described with respect to a particular example of the system 200. However, in other examples, other types of components or controls are implemented. For example, in places of the dual MOSFET trigger switch 204, another type of controllable electronic switch may be used. Similarly, the LED driver 210 may pulse the LEDs using another suitable driving scheme (e.g., at a different frequency and/or duty cycle).

FIG. 3 shows an example of hardware that can be used to implement a light emitting diode (LED) driving circuit 300 in accordance with some embodiments of the disclosed subject matter.

As shown in FIG. 3, an LED driver circuit 300 can include MOSFETS 302 and resistors 304. The LED driver circuit 300 is an example of the LED driver 210 of FIG. 2A. FIG. 3, panel (a), shows a 7.4 volt (V) power supply 306, which can be implemented using 2 lithium batteries in series. FIG. 3, panel (b) shows 3 LEDs 308 of different colors for the NIRS approach. FIG. 3, panel (c) shows control signals handled by a microcontroller 310.

To meet the power consumption rating of the LEDs 308 and the ability to measure the light from the detection side, an LED driver circuit 300 can be implemented based on a configuration of MOSFETS 302 and resistors 304 (e.g., as shown in FIG. 3). In some embodiments, a processor (e.g., a microcontroller 310) can be configured to pull in a high level gate voltage (3.3 V) of an N-channel MOSFET, causing the MOSFET's drain-source resistance to drop to a negligible value. This can be followed by current flow from the positive terminal of the power supply (7.4 V) through the LEDs 308. The current can be limited by a resistor to connect the system to the ground. The resistance values can be calculated to establish the maximum current ratings for each LED color channel. In some embodiments, the resistors 304 can be tuned to acquire a similar output of the photocurrent at the photodiode. Each color output channel can be set to the same bit value to avoid over- and under-saturation issues.

FIG. 3 illustrates and is described with respect to a particular example of the LED driver circuit 300. However, in other examples, other types of components or controls are implemented. For example, different types or sizes of MOSFETS 302 and resistors 304 may be used in some examples, and a power supply of a different voltage may be used in some examples.

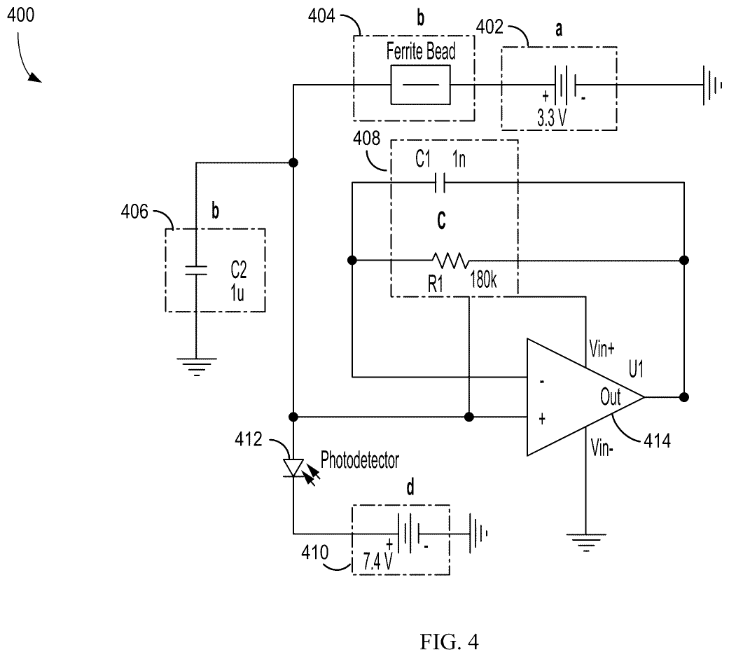

FIG. 4 shows an example of hardware that can be used to implement a transimpedance amplifier (TIA) 400 in accordance with some embodiments of the disclosed subject matter. The TIA 400 is an example of the transimpedance amplifier 214 of FIG. 2A. The TIA 400 can be used to convert light changes detected by a photodiode into readable voltage values for output to an ADC (e.g., the ADC 218 of FIG. 2A), whose output may then be provided to a microcontroller (e.g., the microcontroller 212).

FIG. 4, panel (a) shows a 3.3 V supply 402 from a voltage regulator; FIG. 4, panel (b) shows a ferrite bead 404 and decoupling capacitor 406 that can stabilize the power supply signal; FIG. 4, panel (c) shows a lowpass filter 408 to smooth the signal; and FIG. 4, panel (d) shows power supply 410 (e.g., from one or more lithium batteries and/or power supply 222 of FIG. 2A). In some embodiments, as illustrated in FIG. 4, the TIA 400 can include: (i) a low-noise and low quiescent current, precision operational amplifier 414 (e.g., an OPA376, available from Texas Instruments, Inc), (ii) a combination of gain-setting resistors, (iii) a feedback capacitor, and (iv) a ferrite bead 404. The output of the operational amplifier 414 (also referred to as an op-amp 414) may be provided as the output of the TIA 400 (e.g., which may be received by the ADC 218). The gain-setting resistors and feedback capacitor may be part of the low pass filter 408. A light sensor of the device (e.g., photodiode 412) can be reserved biased by 4.1 V. This voltage value can obtained from the power drop due to the interaction between the two system batteries 410 (7.4 V) and a voltage regulator 402 (3.3 V). The anode from the photodiode 412 can be electrically coupled (e.g., directly connected via a wire and/or other conductive material, such as a wire trace of a circuit board) to the input of the TIA to ensure a current flow from the photocurrent toward the main circuit. A large feedback resistor (e.g., R1=180 kiloohms (kΩ)) may be included to obtain readable voltages, considering current values of 1 microampere (μA) and that the output of the TIA can produce values greater than 0.18 V. Since the inverting input of the system can be biased to the microcontroller voltage (3.3 V), the circuit generates an output of 0 V in an absence of light detection. To reduce noise and smooth the output signal, a capacitor (C1=1 nanofarad (nF)) can be connected in parallel as a low-pass filter. This can attenuate analog high-frequency signals and provide stability to the analog system. In terms of the cutoff frequency design of the low-pass circuit, the system can be configured to only attenuate those signals above the spectra of interest. For example, although human PPG signals do not have a considerable spectrum above 10 Hz, systems implemented in accordance with mechanisms described herein can be configured to operate in pulse mode (e.g., using pulses of 15 ms). Finally, to enhance the stability of the TIA 400, a bypass capacitor 406 (C2=1 microfarad (μF)) and a 1.5 k ferrite bead 404 can be used to decouple the power supply signal.

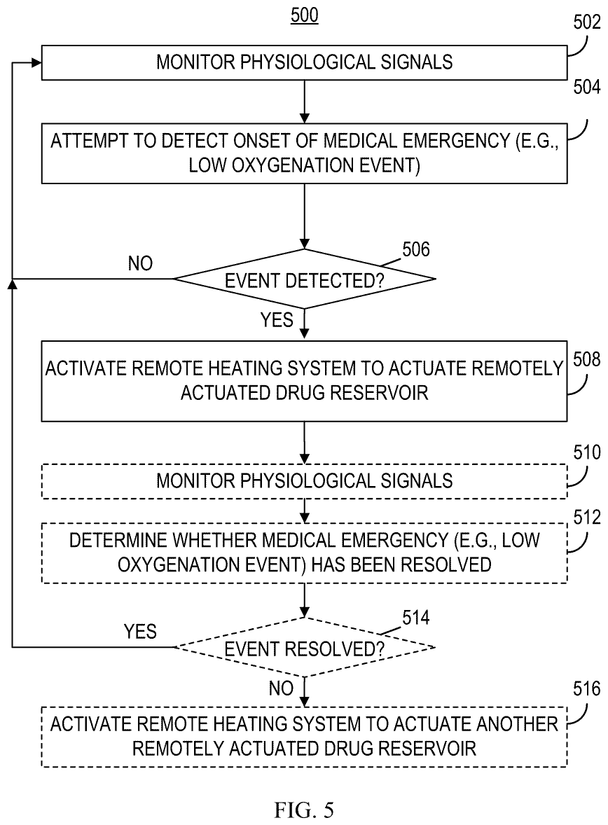

FIG. 5 shows an example of a process 500 for mobile detection of a medical condition and wireless actuation of a drug delivery device in accordance with some embodiments of the disclosed subject matter.

In some embodiments, at block 502, the system 100 can monitor one or more physiological signals. For example, the wearable device 102 can receive output from a physiological monitor 106 (e.g., a photodiode or an NIRS sensor 202, an output of the NIRS sensor 202, etc.) at the processor 104 (e.g., the microcontroller 212), and analyze the output to estimate one or more physiological signals, such as HbO₂, HbR, and/or SpO₂ (e.g., as described above in connection with EQS. (1)-(3)). For example, in some embodiments, the wearable device 102 (e.g., the processor 104) can receive signals from the NIRS sensor described above in connection with FIG. 2A (e.g., via an ADC). For example, in some embodiments, the wearable device 102 (e.g., the processor 104) may drive a light emitting diode (or diodes) of the NIRS sensor (see, e.g., diodes 308 of FIG. 3); may sense, with a photodetector of the NIRS sensor, light output by the LED of the NIRS sensor (see, e.g., photodetector 412 of FIG. 4); and generate the signals to be output by the physiologic sensor based on the light sensed with the photodetector (see, e.g., output of the op-amp 414 in FIG. 4).

At block 504, the system 100 can attempt to detect an onset of a medical emergency (or any other suitable triggering event) that may necessitate timely administration of a drug that is available via a remotely actuated drug reservoir 208. For example, the processor 104 can determine, based on a physiological signal and/or a combination of physiological signals received from the physiological monitor 106, that a low oxygenation event is occurring, potentially due to an opioid overdose.

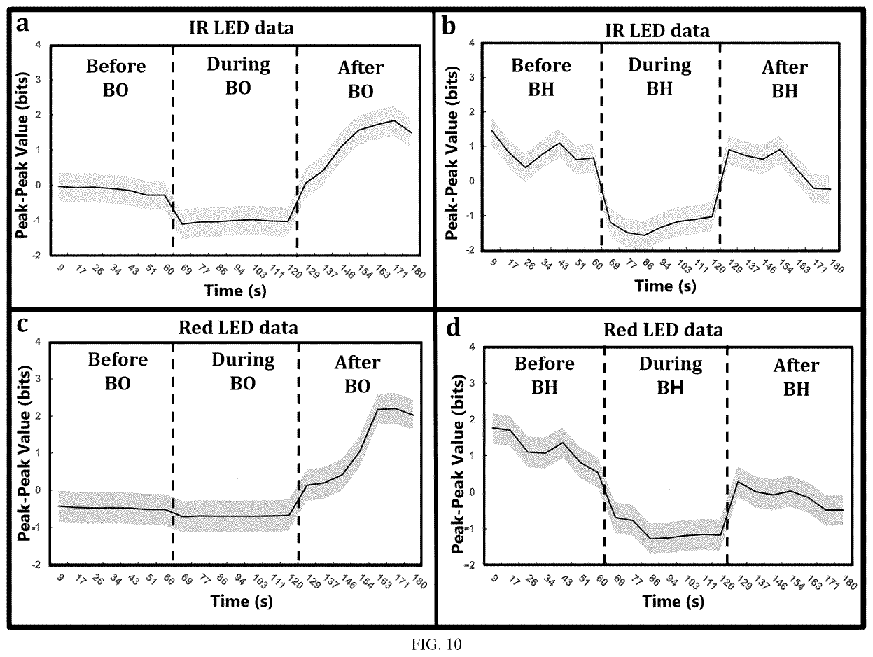

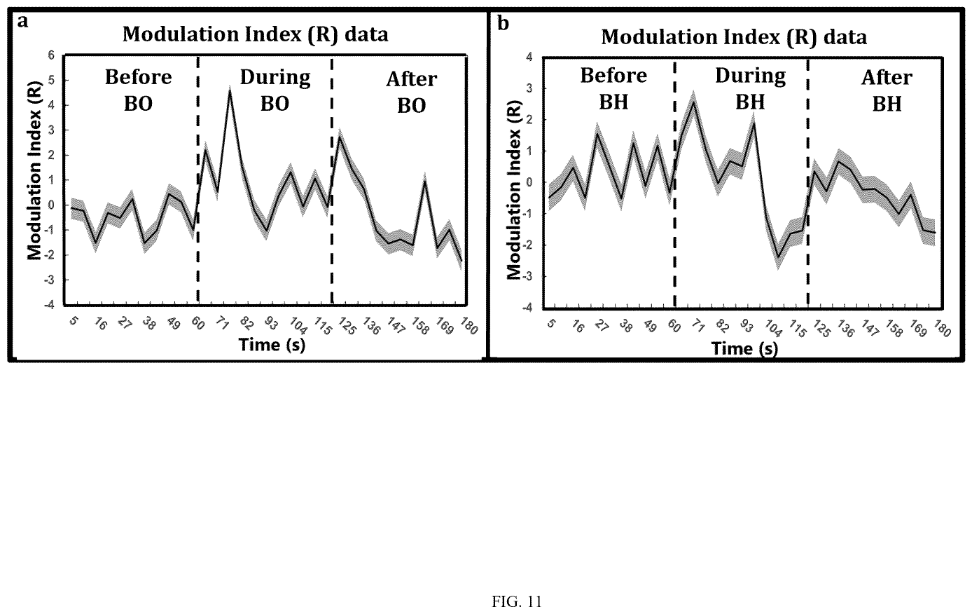

For example, if processor 104 determines that SpO₂ has fallen below a threshold (e.g., indicated by a value of R increasing by a threshold amount) for a predetermined amount of time, processor 104 can determine that a low oxygenation event is occurring (e.g., as described below in connection with FIG. 11).

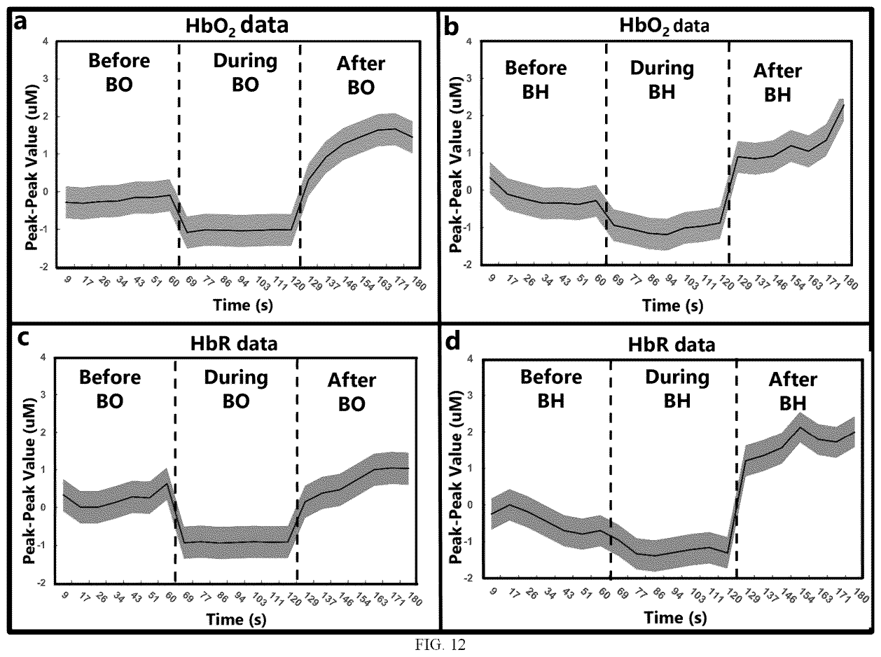

As another example, if processor 104 determines that HbO₂ and/or HbR has decreased by a threshold amount for a predetermined amount of time, processor 104 can determine that a low oxygenation event is occurring (e.g., as described below in connection with FIG. 12).

In some embodiments, processor 104 can use other physiological signals to determine whether a medical emergency is occurring, such as signals from a remote physiological monitor. Such signals can be used, for example, to corroborate symptoms of an ongoing opioid overdose. In some examples, the system 100 includes additional types of sensors to facilitate detecting a medical emergency (e.g., a low oxygenation event). For example, the physiological sensor(s) 106 of the wearable device 102 may further include an inertial motion unit (IMU) configured to measure a position or movement of a user. The processor 104 may determine or infer, based on an output of the IMU, whether the user of the wearable device 102 is motionless (e.g., moving below a threshold amount), has limited movement, is upright, has fallen to the ground, is moving above a threshold amount, etc. The processor 104 may use this determination based on the IMU output as an additional source of sensor information to augment the information provided by the NIRS sensor 202. For example, the processor 104 may detect a medical emergency in response to both detecting a low oxygenation event based on the output of the NIRS sensor 202 (as described above) and that the user is motionless or has fallen based on the IMU output. In contrast, if the processor 104 suspects a low oxygenation event based on the output of the NIRS sensor 202, but the IMU output indicates to the processor 104 that the user is mobile and upright (e.g., walking), the processor 104 may determine that user is not actually suffering from a low oxygenation event. As another example, the wearable device 102 may include a microphone or auditory sensor to sense sound from a user that may be used in a similar manner to corroborate or dismiss a suspected low oxygenation event. Such additional sensor information used for corroboration can reduce false positive detections of medical events by the wearable device 102.

At block 506, if processor 104 determines that a medical emergency is not occurring (“NO” at 506), process 500 can return to block 502 and the system 100 (e.g., the processor 104 of the wearable device 102) can continue to monitor physiological signals from the physiological sensor(s) 106. Otherwise, if processor 104 determines that a medical emergency is occurring (“YES” at block 506), process 500 can move to block 508, and processor 104 can activate a remote heating system 112 to actuate a remotely actuated drug reservoir 208 to dispense drugs (e.g., subcutaneously).

For example, as described above in connection with FIGS. 2A and 2B, processor 104 can cause a signal to be transmitted to a trigger switch (e.g., trigger switch 108, the dual MOSFET trigger switch 204, etc.) to cause the switch to provide power to a remote heating system (e.g., remote heating system 112, the ZVS circuit 206 and coil 224, etc.). Furthermore, in some examples, the process 500 involves the system 100 generating an alarm an audible, tactile, and/or visual alarm (e.g., via display 116) to indicate when an event is detected at block 506. Such an alarm can alert the user and/or nearby individuals in advance of or in parallel with dispensing of drugs via the remotely actuated drug reservoir 208. The alarm may be audible, tactile, or visual. For example, a speaker on or in communication with the wearable device 102 may generate an alert sound (e.g., loud and/or intermittent sounds or beeps), a vibration device on or in communication with the wearable device 102 may generate an alert vibration, and/or a light device on or in communication with the wearable device 102 (e.g., one or more light emitting diodes, the display 116, etc.) may generate an alert light (e.g., bright and/or flashing light(s)). Additionally or alternatively, the wearable device 102 may transmit a message (e.g., via the communication network 130) to cause a phone call, text, or other communication to emergency personnel or services and/or a person of choice (e.g., indicated via emergency contact information that a user has previously stored in the memory 110).

In some embodiments, at block 510, processor 104 can continue to monitor one or more physiological signals. For example, processor 104 can receive output from a physiological monitor (e.g., a photodiode 412 or an NIRS sensor 202, an output of the NIRS sensor 202, etc.), and analyze the output to estimate one or more physiological signals, such as HbO₂, HbR, and/or SpO₂ (e.g., as described above in connection with EQS. (1)-(3).

At block 512, processor 104 can attempt to detect whether the medical emergency (or any other suitable triggering event) detected at block 504 has been resolved. For example, processor 104 can determine, based on a physiological signal and/or a combination of physiological signals, whether a low oxygenation event is still occurring after actuation of the drug delivery reservoir 208 at block 508.

For example, if processor 104 determines that SpO₂ has risen above a re-oxygenation threshold and/or is improving at a threshold rate (e.g., indicated by a value of R decreasing by a threshold amount) for a predetermined amount of time, processor 104 can determine that a low oxygenation event has been resolved.

At block 514, if processor 104 determines that the medical emergency has been resolved (“YES” at block 514), process 500 can return to block 502 so as to cause the processor 104 to continue to monitor physiological signals, or can end.

Otherwise, if processor 104 determines that the medical emergency has not been resolved (“NO” at 514), process 500 can move to block 516, and the processor 104 can activate the remote heating system 112 to actuate another remotely actuated drug reservoir 208 to dispense drugs (e.g., subcutaneously).

For example, as described above in connection with FIGS. 2A and 2B, processor 104 can cause a signal to be transmitted to a trigger switch (e.g., trigger switch 108, the dual MOSFET trigger switch 204, etc.) to cause the switch to provide power to the remote heating system (e.g., remote heating system 112, the ZVS circuit 206 and coil 224, etc.). In some embodiments, multiple drug reservoirs 208 can be implanted, with a first reservoir configured to release a drug after a single actuation of the remote heating system 112, a second reservoir configured to release a drug after multiple actuations of the remote heating system 112, etc.

In some embodiments, one or more of blocks 510-516 can be omitted. For example, if only a single drug reservoir 208 is available, one or more of blocks 510-516 can be omitted.

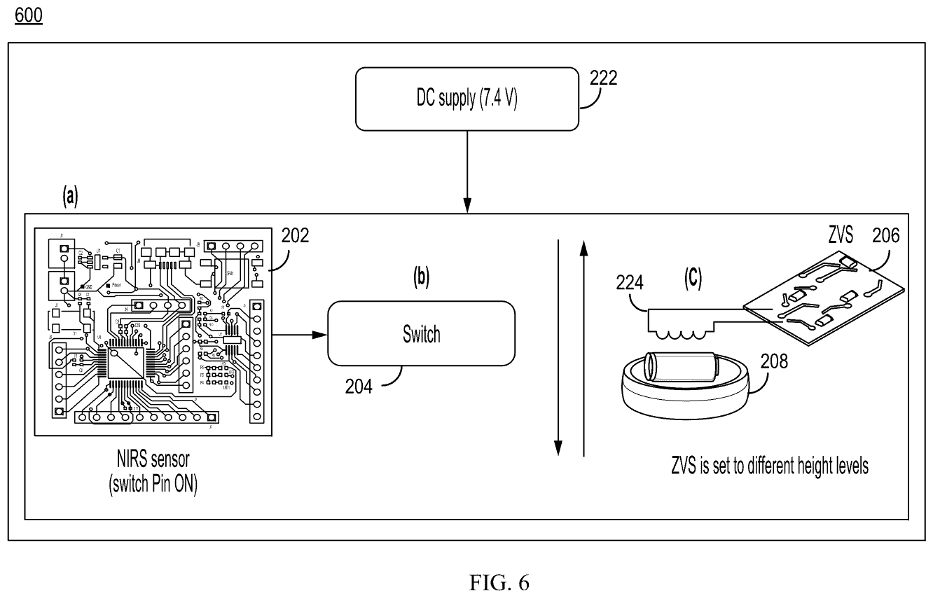

FIG. 6 shows an example 600 of components implemented for a benchtop experiment to test functionality of a wearable device implemented in accordance with some embodiments of the disclosed subject matter.

FIG. 6 shows a block diagram of components implemented for benchtop experiments described herein, including in panel (a), a schematic of the front side of the NIRS sensor 202. For example, a digital pin of the microcontroller 212 is activated to drive the switch 204 and the ZVS circuit 206. FIG. 6, panel (b), shows a representation of the MOSFET switch driver 204 to integrate the instrumentation circuit to the ZVS driver 206. FIG. 6, panel (c) shows a diagram of an electromagnetic field generator, placed at different height levels to melt a phase change material (PCM) of a remotely actuated drug reservoir 208 (e.g., which can be implanted to deliver the drug subcutaneously).

To test the functionality of the wearable device, a straightforward benchtop experiment was implemented to evaluate the performance of the ZVS circuit and how the NIRS sensor 202 activates the entire system 600. FIG. 6 illustrates several components involved in the experiment. A high-level voltage was set to a digital pin on the microcontroller 212 to manually activate the Dual MOSFET switch 204 and the ZVS circuit 206. All components were powered using a DC power supply 222 (e.g., an Agilent E366454A DC Power Supply, available from Agilent Scientific Instrument, Inc).

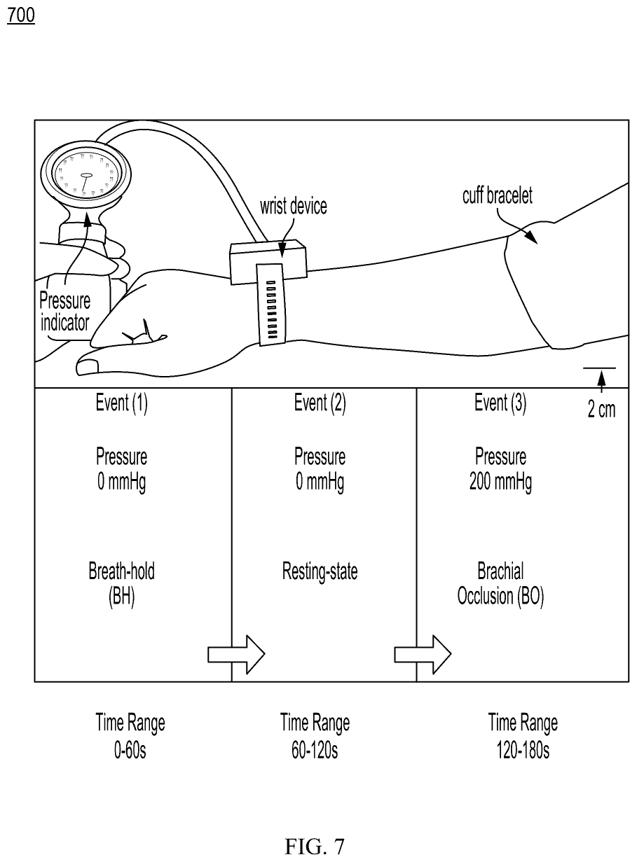

FIG. 7 shows an example of a setup for a human experiment to test capabilities of a prototype wearable device (e.g., the wearable device 102) implemented in accordance with some embodiments of the disclosed subject matter.

FIG. 7 shows a setup 700 for a brachial occlusion trial mimicked with a conventional sphygmomanometer. Signals from the prototype wearable device were recorded during multiple events, including event (1): a low-oxygen event due to breath hold section for a range of 60 seconds; event (2): resting time for 60 seconds; and event (3): low oxygen event due to brachial occlusion at 200 millimeters of mercury (mmHg) of pressure induced through an inflatable cuff. The scale bar of FIG. 7 indicates 2 cm.

To evaluate the capabilities of the NIRS sensor to detect apnea events and cardiorespiratory depression, a test based on brachial artery occlusion was implemented using a conventional sphygmomanometer with a total of 8 volunteers. The human subject research was approved by Purdue University's Institutional Review Board (Protocol #IRB-2020-297). To gather the subjects, criteria of participation was established for the test as follows: (i) the participant must be at least 18 years old or older; (ii) the participant must have a no history of any cardiovascular disease or events that compromise the regular functionalities of the respiratory system; (iii) the patient cannot be an opioid user; and (iv) the patient must be conscious and free or any hallucinogens, substances, or medication that compromise the cognitive system during the test.

The subjects were tested six times for a total of 18 occlusion events. During the trial, volunteers were asked to sit comfortably, keeping the right arm and the feet well supported. Both feet rested flat on the floor and the right hand was placed over a table as indicated in FIG. 7. The prototype wearable device was tightened to a proper level and the inflatable cuff was placed over the upper part of the arm to point out the brachial artery.

Each test included 3 main events with a total duration of three minutes. Event (1): low oxygen state induced by a breath hold (BH) test. In this event, the subject was asked to hold his/her breath continuously for 60 s. However, in case of discomfort or adverse events, the subject was allowed to take as many breaths as needed and resume the trial until the timer reached the total duration of the trial. Event (2): resting time. During this part of the trial, the subject rested for 60 s, breathing without forced episodes. Event (3): low oxygen state induced by a brachial occlusion test. In the final part of the trial, brachial occlusion (BO) was caused due to a continuous pressure of 200 mmHg on the bracelet cuff for 60 s. After the test was completed, the pressure over the arm was released.

FIGS. 8A-F shows example components of the prototype wearable device shown in FIG. 7, and the prototype wearable device worn by two different subjects.

FIG. 8A shows a front side of an NIRS PCB sensor (e.g., NIRS sensor 202), including a TIA (e.g., the TIA 214), a microcontroller (e.g., the microcontroller 212), a 16 bit ADC (e.g., the ADC 218), and supplementary subcircuits. FIG. 8B shows a back side of the NIRS PCB sensor, containing the principal optical component of the device (e.g., LED driver 210), denoted by the sensing area enclosed by the white square. FIG. 8C shows a commercial MOSFET switch driver (e.g., the DUAL MOSFET trigger switch 204) without the wire-to-board header components. FIG. 8D shows a modified ZVS board (e.g., the ZVS circuit 206 as described above in connection with FIGS. 2A and 2B). FIG. 8E shows a view of all the electronic parts of the prototype wearable system packed inside a wrist case worn by a first subject. FIG. 8F shows a final version of prototype wearable device on a second subject. All the scale bars represent 5 mm.

FIGS. 8E-F shows a prototype of a wearable device as described herein (e.g., wearable device 102), including electronics and a prototype version of a wrist case. FIG. 8A shows the front side of the PCB of the NIRS sensor, characterized by the inclusion of the principal electronics of the system as follows: a microcontroller (SAMD21G18A), the implemented design of the TIA circuit, a 3.3 V voltage regulator, and a 16-bit ADC (ADS1115). Supplementary electronic components such as a push button to reset the system, a ferrite bead along with a capacitor to decouple the power supply signal, two female plug connectors to connect two lithium batteries, and a set of resistors, capacitors, and an operation amplifier (OPA376) are included in the top side of the PCB in FIG. 8A. The back side of the PCB NIRS sensor is shown in FIG. 8B, which includes the LED driver (SFH7050). Note that the PCB includes 2 layers, where GND and power supply lines are fully connected through the layers.

To couple the instrumentation circuit (FIGS. 8A-B) with the high-power electronic (FIG. 8D), a commercial MOSFET driver switch was modified. FIG. 8C depicts the commercial MOSFET driver switch with an absence of the header-wire-to-board, with the aim of the reducing the size of switch to facilitate the packaging of the entire components in a 3D printed wrist case (59 mm×49 mm×25 mm).

FIG. 8D shows the modified ZVS driver with a volume of 32 mm×41 mm×3 mm, which is 26% smaller than a commercially available electromagnetic field generator described in Dhowan et al., “Simple minimally-invasive automatic antidote delivery device (a2d2) towards closed-loop reversal of opioid overdose.” The modified ZVS reached 15 kiloamperes per meter (kA/m) of magnetic field at 195 kHz, delivering the necessary power capabilities. FIGS. 8E-F show the integrated electronic components of the system, packed, and placed inside the wrist case. Note that FIGS. 8E-F illustrate two different subjects to validate the wearability of the device, considering different wrist sizes. The wrist of the first subject in FIG. 8E has a perimeter of approximately 196 mm and the wrist of the second subject in FIG. 8F has a perimeter of approximately 216 mm. Both subjects did not exhibit any signs of discomfort while wearing the wrist case.

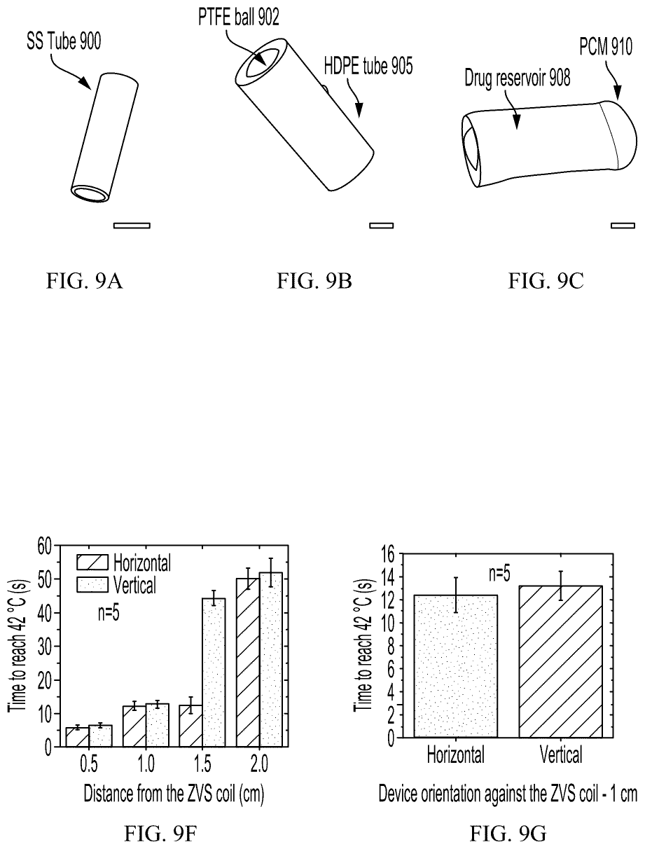

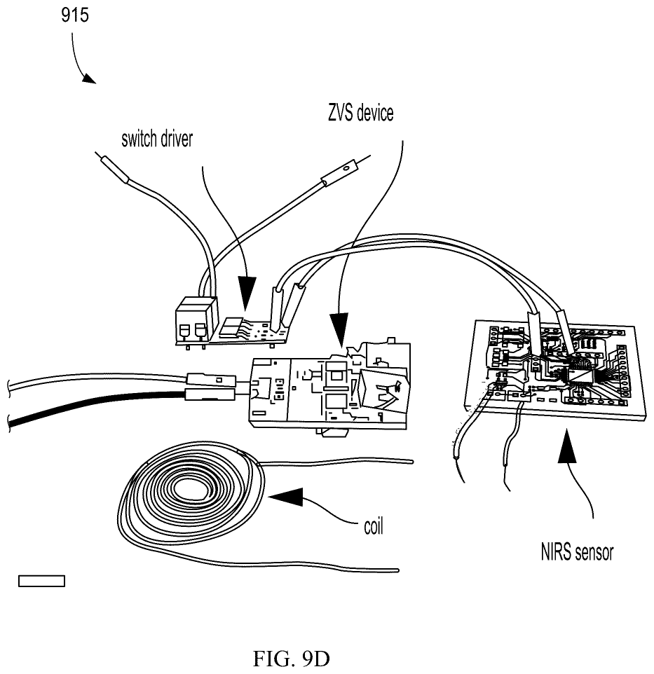

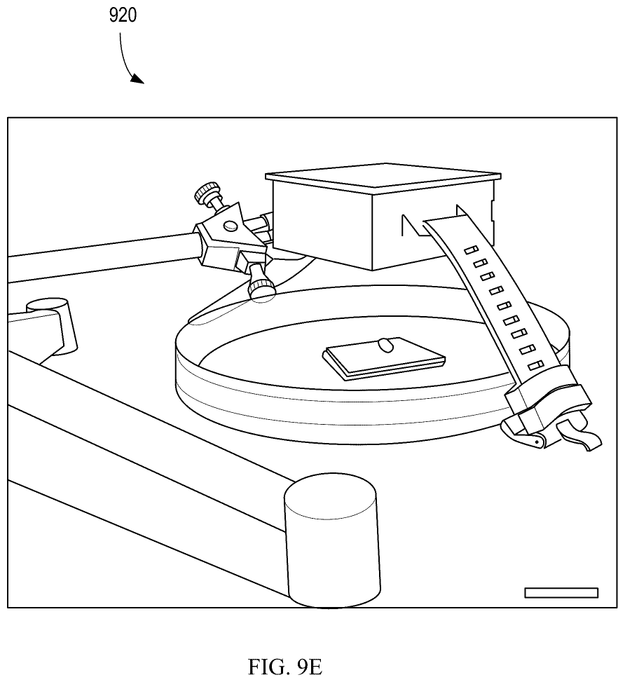

FIGS. 9A-E shows an example of a setup for benchtop experiments to test functionality of the prototype wearable device shown in FIG. 7. FIGS. 9F-G also shows results of the benchtop experiments. FIG. 9A shows a stainless steel (SS) heating element tube 900; FIG. 9B shows a high-density polyethylene (HDPE) tube 902 and a polytetrafluoroethylene (PTFE) sealing ball 905; FIG. 9C shows a prototype of the drug reservoir 908 sealed with PCM 910; FIG. 9D shows electronic components 915 used during the benchtop experiment; FIG. 9E shows a setup 920 of the wearable device to test the ZVS driver at different high levels above the drug delivery capsule; FIG. 9F shows time for the implantable drug delivery device to reach 42 degrees Celsius (°C) from the electromagnetic field generator at different distances; FIG. 9G shows time for the implantable drug delivery device to reach 42 °C from the electromagnetic field generator at a distance of 1 cm. The scale bar for FIG. 9A-C indicates 1 mm, and for FIGS. 9D-E, the scale bar represents 1 cm and 2 cm, respectively.

The benchtop experiments demonstrated the drug release capability of an example of the wearable device 102. FIGS. 9A-C illustrate the used implantable device made of four fully commercial biocompatible elements: the SS heater tube 900, the HDPE tube 902, the PTFE ball 905, and the cover side made of PCM 910.

To demonstrate the drug release, the drug reservoir was activated at different orientations and heights. FIG. 9D depicts electronics components used for the experiments, before packaging inside the 3D printed case. FIG. 9E shows the configuration of the wearable device at a height level of above the drug delivery capsule in a horizontal position.

FIGS. 9F-G depict the quantitative data for the characterization of the heating element. FIG. 9F illustrates the time taken by the device to induce 42 °C over the implantable drug reservoir to different positions and height levels. As expected, smaller distances between the coil and the heater element result in a shorter time to activate the PCM melting point. At 0.5 cm, it required 5.84±0.82 s horizontally and 6.48±0.76 s vertically (n=5, each). As the distance between the coil and the heater increases, the time to reach the proper temperature. At 1 cm away, for the horizontal orientation the implantable capsule required 12.28±1.38 s to reach the 42 °C while the vertical approach took 12.8±1.15 s to achieve the same temperature.

In terms of the drug delivery, the device is capable of ensuring a rapid delivery of a burst of the life-saving antidote. For both vertical and horizontal alignments (5.84 s vs 6.84 s at 0.5 mm distance), the system can reach the appropriate temperature (42 °C) to melt the PCM on time, allowing the delivery of the drug relatively quickly. Also, note that the fast response for the horizontal orientation of the implantable capsule may be due to nonaxial components of the electromagnetic field induced by the ZVS circuit, causing eddy currents over the implantable capsule. Indeed, such a fast-heating process is feasible since a quick response is desirable during an opioid overdose.

FIG. 10 shows examples of peak-peak values of infrared and red intensity recorded during the human experiment described in connection with FIG. 7. More particularly, FIG. 10 shows peak-peak values of IR and Red intensity for the 8 subjects during the BH and BO trials. All graphics are normalized with a 95% interval of confidence indicated by the shade regions in each plot. FIG. 10, panel (a) shows IR intensity peak-peak values for the BO trial; FIG. 10, panel (b) shows IR intensity peak-peak values for the BH trial; FIG. 10, panel (c) shows red intensity peak-peak values for the BO experiments; and FIG. 10, panel (d) shows red intensity peak-peak values for the BH experiment.

FIG. 10 shows the average normalized results of the intensity of red and IR lights across the 8 subjects for the BH and BO trials with a 95% confidence interval denoted by the shaded regions in each plot. FIG. 10, panel (a) displays the peak-peak values for the intensity of the IR LED data before, during, and after the BO experiment, presenting a minimum peak-peak value of −1 during the experiment and a maximum rise of 2 after releasing the brachial occlusion. A similar trend is depicted in FIG. 10, panel (b) for the BH experiment, presenting evidence of a slight decrease to approximately −1.5 in the intensity of the IR light during the test, followed by an increase in the absorption of the light, reaching values greater than 1. Note that before the BH trial, the IR intensity values reached a maximum initial value of approximately 1.6. In regard to the red intensity behavior during the trials, a slight general change compared to the IR trends is observed in FIG. 10, panels c) and (d). For the BO experiment, before and during the trial, a steady negative value of the peak-peak value is evidenced just before the end of the trail (t=118 s). After this, the red intensity values reached positive values greater than 2 until a slight drop as evidenced in FIG. 10, panel (c). Concerning the BH experiment for the red intensity results (FIG. 10, panel (d)), the highest values of the peak-peak red intensity were noticed before the trial (1.8), followed by a steady decrease in those values to −1.5. After the BH, a smooth increase in the peak-peak values is observed, bouncing between +1 and −1.

FIG. 11 shows examples of averaged R modulation index values recorded during the human experiment described in connection with FIG. 7. In FIG. 11, averaged normalized R values for the 8 subjects for the BO and BH experiments with a 95% confidence interval str denoted by the shaded regions in each plot. FIG. 11, panel (a) shows R values before, during and after the BO trial; and FIG. 11, panel (b) shows R data before, during and after the BH trial.

To analyze and calculate changes in HbO₂ and HbR data during the BH and BO trials, the R value is analyzed. FIG. 11 shows the normalized values with a 95% confidence interval highlighted by the shaded regions for the BH and BO experiments. FIG. 11, panel (a) shows the results for R before, during and after the BO test, representing values between −2 and 2 during the resting time and reaching values of 4 during the induced low oxygenation event induced. Note that after the BO test there is a steady decrease in the R values. However, an increase in the R values was expected as a result of the relationship between this parameter and the level of oxygenation in the tissue. Similarly, for the BH trail, the R (FIG. 11, panel (b)) represents a noticeable decrease to −3, indicating a low level of oxygenation in the tissue. It was followed by an increase of the R increase value to positive values, denoting a reoxygenation during resting time.

As described above, the modulation index (R) can be used as a parameter to calculate SpO₂ through the implementation of an NIRS system. Results depicted in FIG. 11, panels (a) and (b) show a slight increase in the R values during the BO and BH tests, validating a physiological expectation for low peripheral oxygenation. Note that high values of R are associated with low levels of SpO₂. FIG. 11, panel (b) shows a sharp decrease in the R values towards the end of the breath hold period. Such decrease can be attributed to brief exasperated breaths taken by participants before the end of the intended breath hold period.

FIG. 12 shows examples of peak-peak values of oxyhemoglobin and deoxyhemoglobin changes calculated during the human experiment described in connection with FIG. 7. More particularly, in FIG. 12, peak-peak values of HbO₂ and HbR changes are shown for the 8 subjects during the BH and BO trials. All graphics are normalized with a 95% interval of confidence indicated by the shade regions in each plot. FIG. 12, panel (a) shows HbO₂ peak-peak concentration values for the BO trial; FIG. 12, panel (b) shows HbO₂ peak-peak concentration values for the BH trial; FIG. 12, panel (c) shows HbR peak-peak concentration values for the BO experiments; and FIG. 12, panel (d) shows HbR peak-peak concentration values for the BH experiment

FIG. 12 illustrates the changes in the HbO₂ and HbR for the BO and BH experiments. Data were averaged and normalized with a 95% confidence interval indicated by the shaded region. FIG. 12, panel (a) presents a slight decrease in the HbO₂ data to −1.5 (micro molar) μM during the BO trial, followed by a rise in the changes to 2 μM approximately after the experiment. It is also possible to observe that changes in HbO₂ concentration are followed by a slight decrease in HbR values because of the presence of an induced low-oxygen event associated with the BO. A similar trend is observed in FIG. 12, panel (b) for BH, where a slight drop of −1.5 μM in HbO₂ values during BH is appreciated, followed by a prominent increase of 3 μM, which might indicate a reoxygenation period. Alike the HbO₂, the HbR changes data depicted, a similar trend for both the BO and BH trials are displayed in FIG. 12, panel (c) and FIG. 12, panel (d), respectively. During the BO trial (FIG. 12, panel (c)), the changes for HbR reached negative values (ranging between −0.5 and −1.3 μM), until the trial was finished. After that, an increase in those values to positive changes to 1.2 μM is shown. Lastly, for the HbR data in FIG. 12, panel (d), following a trend similar to FIG. 12, panel (c), but with more noticeable rising values after the BH trial, reaching positive changes close to 2 μM.

To compare the statistical significance of the data among the groups of interest for the brachial occlusion, prebreath hold, and breath hold experiments, a one-way ANOVA Games-Howell test was conducted with a significance level (α=0.05). Three groups of interest were defined for each set of data containing these variables as follows: (1) red LED data bits, (2) IR LED data bits, (3) HbO₂ calculated values, (4) HbR calculated values, and (5) SpO₂ obtained values for each group. To analyze the data, an open source statistics program (R-4.2.2) was used.

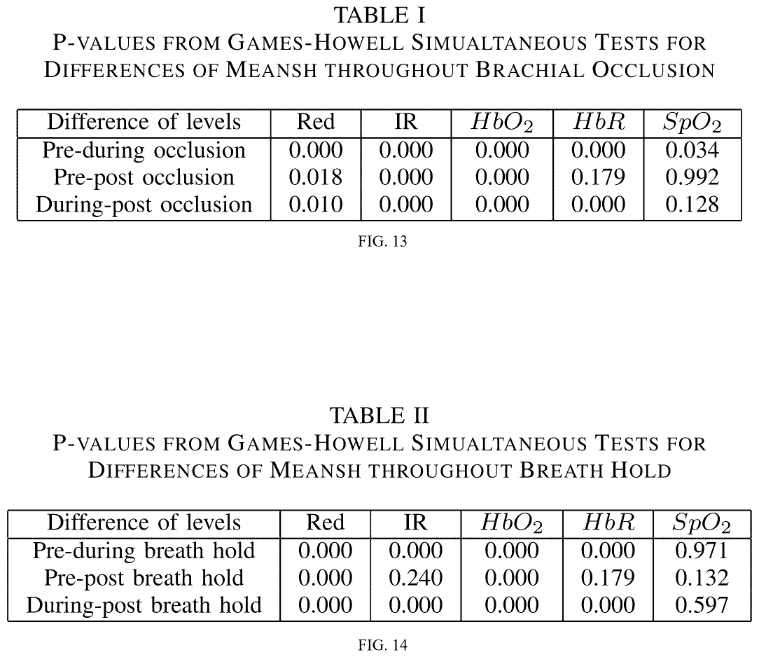

Table 1 and Table 2 in FIGS. 13 and 14, respectively, illustrate a summary of the p-values for the analysis on both the brachial occlusion and breath hold data. The null hypothesis was assumed for no means difference between the groups of interest. Table 1 contains p-values<0.05, except for SpO₂, which was related to the R modulation index for the three stages of the BO experiments. Similarly, in Table 2 most of the p-values were <0.05 besides those related to SpO₂ during the BH experiment, limiting the rejection of the null hypothesis. The groups of interest shown in the tables represent the statistical difference between two stages (e.g., the difference in measurements between the pre-occlusion time period and the occlusion time period is labeled “pre-during occlusion,” while the difference in measurements between the pre-occlusion time period and the post-occlusion time period is labeled “pre-post occlusion,” etc. Using multiple signals can provide redundancy in identifying a real risk event.

In terms of the performance of the NIRS sensor, an important aspect is the ability of the device to detect changes in HbO₂ and HbR concentrations during a simulated opioid-induced overdose event. As described above, a decrease in the concentration of oxyhemoglobin can be expected during brachial occlusion (BO) and breath holding (BH) as indicated by a negative derivative (see, e.g., FIG. 12, panels (a) and (b)). When plotting HbO₂ changes, the negative derivative indicates a consistent negative value owing to an expected change in the Y axis. This phenomenon is associated with low oxygenation in the tissue, leading to a reduction in HbO₂ on the surrounding capillaries. In contrast, HbR concentration values for the BO and BH experiments (see, e.g., FIG. 12, panels (c) and (d)) were predicted to rise by a positive derivative. The physiological reasoning behind such an increase in deoxyhemoglobin and a fall in oxyhemoglobin is based on the event of blood supply cutoff, which prompts muscles to use locally available oxygen. As reoxygenated blood becomes reduced, oxygen supply is slowly depleted as a result of continued muscle use. It is important to note that the possible errors in the data can be explained due to the skin tone on the subjects, which is known as a critical topic for research in the field of optical wearable devices. Another important aspect to consider is noise induced by the movements of the subjects, which can strongly affect the signal obtained during the tests. This aspect can be considered as one of the limitations of our device, where future enhancements in terms of analog and digital signal conditioning need to be performed. On the other hand, during the BH trial, some subjects did not proper hold the respiration for a long period of the test, limiting the accuracy of the obtained data during this test. Nevertheless, having a stable baseline is highly important to better make accurate diagnosis of respiratory depression.

FIG. 13 shows a table that includes a summary of p-values for the analysis on brachial occlusion during the human experiment described in connection with FIG. 7.

For the statistical results for human trials, it is important to highlight the limitation of collecting enough data with proper human subjects testing requirements. However, studies with limited sample size can serve as baselines for future studies to better understand the problem to be addressed and also for the formulation of a feasible statistical study for future approaches. Taking into account the results stated in Table 1 for the BO test, observations in red, IR, HbO₂, and HbR data presented p<0.05, which suggests significant statistical difference between the groups of interest before, during and after the experiment.

Similar statistical results are observable for the pre and post occlusion testing group, except in changes in HbR concentration (p=0.179). This suggests that there is no significant difference in changes in HbR concentration before and post occlusion, which exemplified an ideal outcome as subjects were to simply rest during these periods. The contrary exhibited by the red and changes in IR light intensity data and HbO₂ concentration may be attributed to physical variation among subjects by which their cardiopulmonary system adapts to rapid changes.

FIG. 14 shows a table that includes a summary of p-values for the analysis on breath hold during the human experiment described in connection with FIG. 7.

Similar to the statistical analysis results of brachial occlusion data, breath hold metrics (shown in Table 2) for red, IR, HbO₂ and HbR for the BH test, the p-values obtained for the two comparison groups were <0.05. These outcomes support the expected physiological changes due to reduced oxygenation in the blood. No significant difference was determined before-after breath hold in IR light intensity (p-value=0.24) and consequently, and HbR (p-value=0.179) since the concentration calculations for this value depended on the IR data. Statistical difference among red and IR light intensity data and HbO₂ concentration changes can also be attributed to physiological variation among participants, indicating how the cardiopulmonary system adapts to breathing changes upon induced oxygenation restrictions via breath hold and subsequent influx. Lastly for the statistical analysis, although no statistically significance was found for the SpO₂ groups (Tables 1 and 2), the nuanced difference in measurements for the BH trials (FIG. 11, panel (b)) suggests the ability of the prototype device to provide an acute sensitivity to measure changes in the R values upon low oxygenation events. Similar statistical outcomes for the R values during the BO are depicted in Table 1, except for the pre-during occlusion period (p-value=0.034). It is important to highlight that the overall decrease in R values for the BO test (FIG. 11, panel (a)) indicated increases in the SpO₂ values for both low-oxygenation events.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

It should be noted that, as used herein, the term mechanism can encompass hardware, software, firmware, or any suitable combination thereof.

It should be understood that the above-described steps of the process of FIG. 5 can be executed or performed in any order or sequence not limited to the order and sequence shown and described in the figures. Also, some of the above steps of the process of FIG. 5 can be executed or performed substantially simultaneously where appropriate or in parallel to reduce latency and processing times.

As used herein, the terms “about” and “approximately,” as used herein, should be understandable to one of skill in the art in the context in which they are used. For example, “about” and “approximately” can mean within 10%, within 5%, within 1%, or within 0.5% of the corresponding value. For example, “about 7.4 volts” may mean 7.4 volts+/−10% (e.g., a voltage between 6.66 volts and 8.14 volts).

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by any allowed claims that are entitled to priority to the subject matter disclosed herein. Features of the disclosed embodiments can be combined and rearranged in various ways.

What is claimed is:

1. A system for mobile detection of a medical emergency and wireless activation of a drug delivery device, the system comprising:
a power supply;
a physiological sensor;
a trigger switch;
a signal generator;
a transmitting coil electrically coupled to the power supply via the trigger switch and the signal generator; and
at least one processor that is programmed to:
receive signals from the physiological sensor;
determine that a triggering event has occurred based on the signals received from the physiological sensor; and
in response to determining that the triggering event has occurred, cause the trigger switch to provide power to the signal generator, such that power is transmitted by the transmitting coil to wirelessly activate the drug delivery device.

2. The system of claim 1, wherein the power supply comprises at least one battery, and is configured to provide power at about 7.4 volts.

3. The system of claim 1, wherein the physiological sensor comprises a near-infrared spectroscopy (NIRS) sensor.

4. The system of claim 1, wherein the signal generator comprises a zero-voltage switching (ZVS) circuit.

5. The system of claim 1, wherein the transmitting coil comprises at least one selected from a group of a pancake coil or a polyamide-insulated copper magnetic wire.

6. The system of claim 1, further including a wrist case and a wrist strap, wherein the wrist case houses the power supply, the physiological sensor, the trigger switch, the signal generator, the transmitting coil, and the at least one processor.

7. The system of claim 1, wherein the signals comprise values output by a photodetector of the physiological sensor.

8. The system of claim 7, wherein the at least one processor is further programmed to:
estimate oxyhemoglobin (HbO₂) and deoxyhemoglobin (HbR) based on the values.

9. The system of claim 7, wherein the at least one processor is further programmed to:
estimate a variable R that is indicative of oxygen saturation (SpO₂) based on the values.

10. The system of claim 1, wherein the signals comprise oxyhemoglobin (HbO₂) and deoxyhemoglobin (HbR) values output by the physiological sensor.

11. The system of claim 1, wherein the signals comprise a variable R output by the physiological sensor.

12. A method for mobile detection of a medical emergency and wireless activation of a drug delivery device, the method comprising:
receiving signals from a physiological sensor;
determining that a triggering event has occurred based on the signals received from the physiological sensor; and
in response to determining that the triggering event has occurred, causing a trigger switch to provide power to a signal generator, such that power is transmitted by a transmitting coil to wirelessly activate the drug delivery device.

13. The method of claim 12, wherein causing the trigger switch to provide power to the signal generator includes: providing power, by a at least one battery, at about 7.4 volts to the signal generator.

14. The method of claim 12, wherein the physiological sensor comprises a near-infrared spectroscopy (NIRS) sensor, the method further comprising:
driving a light emitting diode of the NIRS sensor;
sensing, with a photodetector of the NIRS sensor, light output by the LED of the NIRS sensor; and
generating the signals to be output by the physiologic sensor based on the light sensed with the photodetector.

15. The method of claim 12, wherein the signal generator comprises a zero-voltage switching (ZVS) circuit, and in response to power being provided to the signal generator, the signal generator drives the transmitting coil to generate heat to activate the drug delivery device.

16. The method of claim 12, wherein determining that the triggering event has occurred based on the signals received from the physiological sensor includes at least one selected from a group of:
determining that SpO₂ of a wearer the physiological sensor has fallen below a threshold,
determining that HbO₂ of a wearer the physiological sensor has decreased by a threshold amount, or
determining that HbR of a wearer the physiological sensor has decreased by a threshold amount.

17. The method of claim 12, wherein determining that the triggering event has occurred based on the signals received from the physiological sensor includes determining that a low oxygenation event has occurred.

18. The method of claim 12, wherein the signals comprise values output by a photodetector of the physiological sensor.

19. A non-transitory computer readable medium containing computer executable instructions that, when executed by a processor, cause the processor to perform a method for mobile detection of a medical emergency and wireless activation of a drug delivery device, the method comprising:
receiving signals from a physiological sensor;
determining that a triggering event has occurred based on the signals received from the physiological sensor; and
in response to determining that the triggering event has occurred, causing a trigger switch to provide power to a signal generator, such that power is transmitted by a transmitting coil to wirelessly activate the drug delivery device.

20. The non-transitory computer readable medium of claim 19, wherein the physiological sensor comprises a near-infrared spectroscopy (NIRS) sensor.
